(19) United States
(12) Patent Application Publication
(10) Pub. No.: US 2008/0146955 A1
(43) Pub. Date: Jun. 19, 2008

(54) Respiration Sensor, Using Method of Respiration Sensor, and Respiration State Monitor

(75) Inventors: Katsuhiko Horii, Aichi (JP); Atsushi Wakita, Aichi (JP); Hiroyuki Nagatani, Aichi (JP); Junichi Akiyama, Aichi (JP); Tetsuji Tanada, Aichi (JP)

Correspondence Address: SUGHRUE MION, PLLC, 2100 PENNSYLVANIA AVENUE, N.W., SUITE 800, WASHINGTON, DC, 20037, US

(73) Assignee: NGK SPARK PLUG CO., LTD, NAGOYA-SHI (JP)

(21) Appl. No.: 11/885,993

(22) PCT Filed: Mar. 6, 2006
(86) PCT No.: PCT/JP2006/304280

§ 371 Date: Sep. 10, 2007

(30) Foreign Application Priority Data
Mar. 9, 2005 (JP) 2005-065373
Jun. 24, 2005 (JP) 2005-185411

(51) Int. Cl. A61M 25/098; A61M 16/00

(52) U.S. Cl. 600/529; 128/204.23

(57) ABSTRACT

A respiration sensor to be attached on a surface of a living body for detecting a respiration state of the living body comprises a detecting element for outputting a signal corresponding to at least one of the respiration states of breathing and snoring of the living body and a substrate for retaining the detecting element at a predetermined detection position apart from the surface of the living body. The detecting element is arranged so that breath from both a mouth and a nose is brought into contact therewith.

### TECHNICAL FIELD

The present invention relates to a respiration sensor capable of monitoring a respiration state such as, for example normal/abnormal breathing and snoring during sleep by using a piezoelectric film or the like, a method for using the respiration sensor and a respiration state monitor.

### BACKGROUND ART

As conventional technology for monitoring a respiratory state of a person, there is known a technology of sticking a thermistor near a mouth (refer to Patent Document 1).

There is also known technology of detecting motions of a body by using a piezoelectric element and monitoring a respiration state on the basis of the detection results (refer to Patent Document 2).

Further, there is known a technology of detecting breathing by image processing with infrared rays (refer to Patent Document 3).

Still further, in recent years, there has been disclosed technology of sticking PVDF (polyvinylidene fluoride) film on a mouth to detect breathing from the detection signal (Patent Document 4).

Patent Document 1: Japanese Patent Publication No. 2794196 (page 2, FIG. 2)

Patent Document 2; Japanese Patent Publication No. 2803374 (page 1, FIG. 2)

Patent Document 3 Japanese Patent Publication No. 3390802 (page 1, FIG. 1)

Patent Document 4: U.S. Pat. No. 5,311,875 (page 1, FIG. 1)

### DISCLOSURE OF THE INVENTION

### Problems to be Solved by the Invention

However, the technology of Patent Document 1 has a problem that a large error results, depending on the position of the thermistor.

Further, the technologies of Patent Documents 2 and 3 have a problem that an apparatus is made large.

The technology of Patent Document 4 also has a problem that a flat-plate like film is directly stuck on a mouth to result in poor measurement accuracy.

Further, there is also a problem that snoring cannot be detected stably by a simple method.

The present invention has been accomplished in view of the above problems, an object of which is to provide a respiration sensor that suppresses errors, is reduced in size, is capable of easily detecting breathing from a mouth or a nose and also detects a state of snoring, a method for using the respiration sensor and a respiration state monitor.

### Means for Solving the Problem

(1) The invention of claim 1 is characterized in that a respiration sensor is stuck on a surface of a living body to detect a respiration state of the living body, which is provided with a detecting element for outputting a signal corresponding to at least one of the respiration states of breathing and snoring of the living body and a substrate for retaining the detecting element at a predetermined detection position apart from the surface of the living body.

In the present invention, the detecting element is arranged at a predetermined detection position apart from the surface of the living body by the substrate (in other words, at a position where breath from breathing or snoring flows and thus a respiration state can be detected). Thus, as compared with a case where the detecting element is directly stuck on a mouth, it is possible to easily detect the state of breath from a mouth or a nose (at least one of them) and thereby to detect breathing or snoring with high accuracy.

It is noted that the detecting element and the substrate can be made with members different in material but may be made using the same material. In other words, the present invention also covers a respiration sensor in which the substrate is made with the same material as that of the detecting element (for example, that made with the same piezoelectric body) (the same will apply hereinafter).

(2) The invention of claim 2 is characterized in that the detecting element is a piezoelectric element or a temperature sensing element.

The present invention exemplifies the detecting element.

It is noted that any piezoelectric element 18 usable as long as it can be distorted by breathing or snoring to output a measurable signal, including a piezoelectric element provided with an electrode on each side of a plate-form piezoelectric body, in particular a piezoelectric element in which a thin-film electrode is arranged on each side of a film-like piezoelectric body. As such a piezoelectric body, a piezoelectric body using an organic polymer material, for example, PVDF (polyvinylidene fluoride) can be adopted. Further, the temperature sensing element includes a thermocouple, a thermistor and a temperature-measuring resistor, etc.

(3) The invention of claim 3 is characterized in that the detecting element in arranged so as to oppose a breathing direction of a mouth and also extend along a breathing direction of a nose.

In the present invention, a film-like detecting element (for example, a piezoelectric element) which will vibrate or detect heat, for example, on flow of breath, is arranged at a position apart from the surface of the living body so as to oppose a breathing direction of a mouth and extend along a breathing direction of a nose. Therefore, the respiration sensor is less likely to be influenced by factors other than breathing and snoring (for example, movement of a mouth and the like) and is able to detect breathing and snoring with higher accuracy than a conventional sensor.

In this instance, an expression of “oppose a breathing direction of a mouth” means a state that, for example, breath from a mouth is brought into contact with a detecting element at a deep angle greater than a predetermined angle (for example, a state at which the detecting element is arranged so that breath from a mouth is in contact with the element to block the flow of breath). For example, an angle between the breathing direction of a mouth and a direction in which the detecting element extends is in a range of 90°±30°. Further, an expression of “extend along the breathing direction of a nose” means a direction similar to that at which breath flows from a nose. An angle between the breathing direction of a nose and the direction in which the detecting element extends is, for example, in a range of 180°±30°.

(4) The invention of claim 4 is characterized in that the detecting element is arranged so that breath at least either from a mouth or a nose is brought into contact therewith at an angle greater than a predetermined angle.

In the present invention, breath at least either from a mouth or a nose is brought into contact with the detecting element at an angle greater than a predetermined angle (an angle greater than 0°, for example, 10° or greater), thereby, there is provided an advantage that a respiration state can be easily detected.

(5) The invention of claim 5 is characterized in that the substrate is configured so that breath at least either from a mouth or a nose can be guided to the detecting element.

In the present invention, the substrate is formed so that breath from a nose or a mouth is guided to the detecting element, thereby making it possible to detect breathing or snoring with high accuracy.

(6) The invention of claim 6 is characterized in that an inside of the substrate, which is on the living body side, is formed in a concave shape.

In the present invention, the inside of the substrate is formed in a concave shape, thus making it possible to guide effectively breath from a nose or a mouth to the detecting element side and detect breathing or snoring with high accuracy.

(7) The invention of claim 7 is characterized in that the detecting element is arranged on a bottom surface of a concave groove of the substrate.

In the present invention, the detecting element is arranged, for example, inside the strip-shaped central part of the substrate, by which breath from a nose or a mouth can be guided effectively to the detecting element side. Therefore, there is provided such an advantage that measurement can be made with high accuracy.

(8) The invention of claim 8 is characterized in that the concave groove is formed so that a cross section perpendicular to a flow path of breath from a nose gives a trapezoidal shape.

In the present invention, the cross section of the flow path along the concave groove (the flow path of breath from a nose) is formed in a trapezoidal shape (in other words, the mouth side is wider in opening portion), by which breath from a mouth can be guided effectively to the bottom side of the concave groove to improve measurement accuracy.

(9) The invention of claim 9 is characterized in that the concave groove is formed so that the farther an area of the cross section perpendicular to the flow path of breath from a nose is from a nose side, the smaller it is.

In the present invention, the area of the cross section of the flow path along the concave groove (flow path of breath from a nose) is set to be smaller at the leading end side than on the nose side, by which breath from a nose can be effectively collected at the leading end side. Therefore, the detecting element is arranged at the leading end side to increase measurement accuracy.

(10) The invention of claim 10 is characterized in that the nose side of the substrate protrudes further to the nose side than a position at which the substrate is attached to the living body along the flow path of breath from a nose in the concave groove.

Thereby, where the respiration sensor is attached between a mouth and a hose, the substrate can cover the periphery of the nostrils without any space therebetween. It is, therefore, possible to collect effectively breath from a nose and improve measurement accuracy accordingly.

(11) The invention of claim 11 is characterized in that the substrate is a plate-like member (for example, a film-like member).

The present invention exemplifies a constitution of the substrate.

(12) The invention of claim 12 is characterized in that the detecting element is formed in a plate shape and includes a surface opposing a breathing direction of a mouth and also extending along a breathing direction of a nose.

The present invention exemplifies a surface configuration of the piezoelectric element made with film, for example, which is formed in a flat plate or a curved shape.

It is noted that the substrate includes various shapes of members such as those in combination of a film-like member, a mesh-like member and a wire.

(13) The invention of claim 13 is characterized in that the substrate is formed in a plate shape and includes the surface opposing a breathing direction of a mouth and also extending along a breathing direction of a nose.

The present invention exemplifies a surface configuration of the substrate made with film, for example, which is formed in a flat plate or a curved shape.

(14) The invention of claim 14 is characterized in that the detecting element is arranged on a surface of the substrate.

The present invention exemplifies an arrangement of the detecting element. Thereby, the detecting element can be arranged easily at a desired position of the substrate.

(15) The invention of claim 15 is characterized in that the detecting element is arranged on the living body side of the substrate.

In the present invention, the detecting element is arranged on the substrate which is on the living body side (inner side). Therefore, there is provided an advantage that breath is easily in contact with the detecting element directly to realize a higher measurement accuracy.

(16) The invention of claim 16 is characterized in that where the detecting element is a piezoelectric element, a ventilation hole is formed in the substrate according to a position at which the piezoelectric element is arranged.

There is a tendency that change in output of the piezoelectric element is made small according to a small difference in temperature. In contrast, in the present invention, since a ventilation hole is formed in the vicinity of the piezoelectric element, breath which has once reached the piezoelectric element can be immediately discharged outside. Thereby, there is provided an effect that measurement accuracy is improved due to suppressing an increase in temperature of the piezoelectric element.

(17) The invention of claim 17 is characterized in that the ventilation hole is formed at a projection area of the detecting element to the substrate (in this instance, a piezoelectric element).

In the present invention, the ventilation hole is formed at a portion of the substrate (that is, the projection area) outside the piezoelectric element (that is, on the side opposite to the living body). Therefore, breath which has once reached the piezoelectric element is immediately discharged outside. Thereby, there is provided an effect that the measurement accuracy is improved due to suppressing an increase in temperature of the piezoelectric element.

It is noted that the projection area is an area where the piezoelectric element is projected (perpendicularly) with respect to the surface of the substrate.

(18) The invention of claim 18, is characterized in that the ventilation hole is formed beside the detecting element (in this instance, a piezoelectric element).

Thereby, there is provided an effect that measurement accuracy is improved due to suppressing an increase in temperature of the piezoelectric element.

(19) The invention of claim 19 is characterized in that where the detecting element is a piezoelectric element, the substrate is not provided outside the piezoelectric element but the piezoelectric element is exposed outside.

Therefore, breath which has once reached a piezoelectric element is immediately discharged outside. Thereby, there is provided an effect that the measurement accuracy is improved due to suppressing an increase in temperature of the piezoelectric element. There is also provided an effect that the piezoelectric element can be deflected more easily to result in an increased output of the sensor.

(20) The invention of claim 20 is characterized in that the respiration sensor is provided with a plate-like piezoelectric body having a first portion and a second portion, in which the detecting element is constituted with the first portion of the piezoelectric body and electrodes installed on each side of the first portion in a thickness direction, and the substrate is constituted with the second portion of the piezoelectric body.

In the present invention, a plate-like piezoelectric body (including a film-like one) is used as the respiration sensor, by which the respiration sensor can be made thinner.

(21) The invention of claim 21 is characterized in that an opening portion penetrating through the substrate is formed in a face of the substrate opposing a breathing direction of a mouth.

In the present invention, the opening portion is formed on the substrate, thereby there is provided an advantage that breath from a mouth easily flows in the vicinity of the detecting element to improve measurement accuracy.

(22) The invention of claim 22 is characterized in that a slit is provided along an exhaling direction from the leading end at a portion of the substrate where the detecting element is arranged and which is positioned at the leading end side in the exhaling direction of a nose.

In the present invention, a slit is provided at a portion where the detecting element is arranged (for example, respiration detecting section), thereby, a tab-like configuration is provided. Therefore, there is provided an advantage that the detecting element easily vibrates by breath from a mouth or the like to improve measurement accuracy.

(23) The invention of claim 23 is characterized in that the substrate is folded or curved to give a constitution for keeping a distance between the living body and the detecting element.

For example, a line-symmetrical substrate is folded at the symmetrical axis to give a chevron shape (projecting on one side), by which the detecting element can be arranged at a detection position apart from the living body. Thereby, it is possible to easily arrange the detecting element in the above constitution.

Further, for example, a line-symmetrical substrate is folded chevronwise, by which the detecting element can be arranged at a detection position apart from the living body. Thereby, it is possible to easily arrange the detecting element in the above constitution.

(24) The invention of claim 24 is characterized in that the substrate includes a leg for separating the detecting element from the surface of the living body.

For example, the detecting element is arranged at the central part of the substrate, the leg extending laterally is provided, and the leg is allowed to be in contact with the living body, by which it is possible to arrange the detecting element separate from the surface of the living body.

(25) The invention of claim 25 is characterized in that an adhering portion for adhering the respiration sensor to the living body is provided at a leading end side of the leg on the living body side.

In the present invention, the respiration sensor is adhered to the living body at the adhering portion which is the leading end of the leg (adhesive tape or the like), by which the respiration sensor can be easily fixed to the living body.

(26) The invention of claim 26 is characterized in that the respiration sensor comprises a pair of left and right legs, and a connecting portion for connecting these legs and further comprises an adhering portion for adhering the respiration sensor to the living body. The adhering portion is provided on the living body side of the connecting portion.

In the present invention, the adhering portion is provided at the connecting portion for connecting a pair of left and right legs, thereby there is provided an advantage that the respiration sensor can be fixed without fail. Further, the present invention is constituted so that the legs are connected at the connecting portion, thereby the detecting element can be consistently arranged at a definite position.

(27) The invention of claim 27 is characterized in that a piezoelectric element for detecting breathing and a piezoelectric element for detecting snoring are arranged as the detecting element.

The present invention is provided with a piezoelectric element for detecting breathing and a piezoelectric element for detecting snoring, thereby it is possible to detect the presence or absence of breathing or snoring from a signal sent from each of the piezoelectric elements.

It is noted that a signal from the same piezoelectric element is used to detect breathing or snoring from a difference in signal characteristics between breathing and snoring.

However, there is provided an advantage that the measurement can be made with higher accuracy when different piezoelectric elements are used.

(28) The invention of claim 28 is characterized in that the piezoelectric element for detecting breathing is different in thickness from the piezoelectric element for detecting snoring.

For example, the piezoelectric element for detecting snoring is made thinner than the piezoelectric element for detecting breathing. In other words, where the thickness is changed, the piezoelectric element, which is thinner, is more likely to vibrate, thus making it possible to easily detect breathing and distinguish from snoring without fail.

(29) The invention of claim 29 is characterized in that where the detecting element is a piezoelectric element, an electrically-conductive shield layer is arranged so as to cover the surface of the piezoelectric element.

In the present invention, an electrically-conductive shield layer is arranged on the surface of the sensor body, for example, the face on the living body side or the face opposite to the living body side. Therefore, the shield layer is grounded, by which electromagnetic wave can be shielded to decrease electrical noise in response to the piezoelectric element and also prevent electrostatic charge of the sensor. As a result, the measurement accuracy can be improved.

It is noted that the shield layer may be arranged so as not to be in contact with the living body or the piezoelectric element to avoid interference with a signal output from the respiration sensor, or arranged so as to be in contact with one electrode.

In this instance, various types of materials such as metal foils of aluminum or the like and electrically-conductive coating materials may be used as the shield layer.

(30) The invention of claim 30 is characterized in that where the detecting element is a piezoelectric element, a leading end side of the piezoelectric element (the side opposite to a nose) is bent toward the living body or the piezoelectric element is attached to the substrate at a certain angle.

In the present invention, the leading end of the piezoelectric element is bent or curved, or the piezoelectric element is attached to the substrate at a certain angle (an angle exceeding 0°, for example, 10° or more), by which it is erected on the living body side. Thereby, breath from a nose easily makes in contact with the piezoelectric element to improve the measurement accuracy.

(31) The invention of claim 31 is characterized in that the respiration sensor is foldable and developable.

In the present invention, the respiration sensor is foldable or developable on the sensor body. Therefore, at the time of shipment or packaging, the respiration sensor is folded to make it compact and can be handled more easily.

(32) The invention of claim 32 is characterized in that the respiration sensor is provided with a substrate for retaining the detecting element and an outer cover for covering the detecting element outside the substrate.

The present invention is constituted so as to cover the outside of the substrate having the detecting element by the outer cover, thus making it possible to prevent an inadvertent contact of fingers and others with the detecting element. Further, a wiring extended from the detecting element can be arranged between the substrate and the outer cover, thereby there is provided an advantage that the wiring is less likely to cause interference.

(33) The invention of claim 33 is characterized in that a ventilation hole is formed on the outer cover.

Where the detecting element is a piezoelectric element, there is a tendency that change in output of the piezoelectric element is made small according to a small difference in temperature. On the other hand, in the present invention, a ventilation hole is formed in the vicinity of the outer cover, by which breath which has once reached the piezoelectric element can be immediately discharged outside. Thereby, there is provided an effect that measurement accuracy is improved due to suppressing an increase in temperature of the piezoelectric element.

(34) The invention of claim 34 is a method for using the respiration sensor described in claim 31, in which the respiration sensor is folded at the time of shipment or packaging.

The respiration sensor used in the present invention is foldable or spreadable. Thus, at the time of shipment or packaging, the respiration sensor is folded to make it compact and can be handled more easily.

(35) The invention of claim 35 is a method for using the respiration sensor described in any one of claims 1 to 33, wherein the respiration sensor is stuck between a mouth and a nose in using the respiration sensor.

In the present invention, the respiration sensor is stuck between a mouth and a nose, by which breathing from a mouth or a nose and snoring can be easily detected.

(36) The invention of claim 36 is a respiration state monitor for monitoring a respiration state of the living body by using a signal from the respiration sensor described in any one of claims 1 to 33 which is provided with an amplifier section for amplifying signals from the respiration sensor and a storage device for storing the signals.

Therefore, the respiration sensor is connected to the respiration state monitor, by which the respiration state can be easily detected.

(37) The invention of claim 37 is characterized in that a signal from the respiration sensor is discriminated by the frequency band to detect a breathing signal which indicates breathing and a snoring signal which indicates snoring.

The breathing signal obtained from the piezoelectric element due to breathing is different in frequency from the snoring signal obtained from the piezoelectric element due to snoring. It is, therefore, possible to discriminate breathing from snoring by the frequency of the signal concerned.

(38) The invention of claim 38 is characterized in that a portable storage device is attached in a removable manner.

Thereby, data can be handled more easily. It is noted that various types of storage devices such as memory cards (flash cards, SD cards and the like) and CDs may be used as the portable storage device.

(39) The invention of claim 39 is characterized in that the respiration state monitor is provided with communication functions for transmitting data to an outside.

Thereby, data can be handled more easily. It is noted that communication functions include wire and wireless transmission (electric waves, infrared rays and the like).

### BRIEF DESCRIPTION OF THE DRAWINGS

FIG. 1 is a perspective view of a respiration sensor of Embodiment 1.

FIG. 2 (a) is an explanatory view of the front side of the respiration sensor of Embodiment 1, with the sensor body being developed, FIG. 2(b) is an explanatory view of the back side thereof, and FIG. 2(c) is an explanatory view showing schematically the cross section of FIG. 2(a) taken along line A-A.

FIGS. 3 (a) and (b) are explanatory views showing a method for using the respiration sensor of Embodiment 1.

FIG. 4 is an explanatory view showing a respiration state monitor used in embodiment 1.

FIG. 5 is a perspective view of a respiration sensor of Embodiment 2.

FIG. 6 is an explanatory view showing a respiration sensor of Embodiment 3, with the sensor body being developed.

FIG. 7 is a perspective view illustrating a state that a respiration sensor of Embodiment 4 is folded.

FIG. 8 (a) is an explanatory view of the front side of a respiration sensor of Embodiment 5, with the sensor body being developed, FIG. 8(b) is an explanatory view of the back side thereof, and FIG. 8(c) is an explanatory view showing schematically the cross section of FIG. 8(a) taken along line A-A.

FIG. 9 (a) is a perspective view of a respiration sensor of Embodiment 6, and FIG. 9(b) is an explanatory view showing a method for using the respiration sensor.

FIG. 10 is a perspective view of a respiration sensor of Embodiment 7.

FIG. 11 is a perspective view of a respiration sensor of Embodiment 8.

FIG. 12 is a perspective view of the shield layer of the respiration sensor in Embodiment 8.

FIG. 13 is an explanatory view showing a respiration state monitor used in Embodiment 8.

FIG. 14 is a perspective view of a respiration sensor of Embodiment 9.

FIG. 15 is a perspective view of a respiration sensor of Embodiment 10.

FIGS. 16 (a) and (b) are explanatory views showing a method for using the respiration sensor of Embodiment 10.

FIG. 17 is an explanatory view showing schematically the cross section of a respiration sensor of Embodiment 11.

FIG. 18 is a perspective view of a respiration sensor of Embodiment 12.

FIG. 19 is a plan view of the respiration sensor of Embodiment 12.

FIG. 20 (a) is a developed view of the substrate of the respiration sensor in Embodiment 12, and FIG. 20(b) is a side view of the respiration sensor.

FIG. 21 is an explanatory view showing a state that the respiration sensor of Embodiment 12 is attached.

FIG. 22 is a perspective view of a respiration sensor of Embodiment 13.

FIG. 23 is a perspective view of a respiration sensor of Embodiment 14.

FIG. 24 is a perspective view of a respiration sensor of Embodiment 15.

FIG. 25 (a) is a developed view of the substrate of the respiration sensor in Embodiment 15, and FIG. 25(b) is a side view of the respiration sensor.

FIG. 26 is an explanatory view showing a state that the respiration sensor of Embodiment 15 is attached.

FIG. 27 (a) is a perspective view of a respiration sensor of Embodiment 16, FIG. 27(b) is a sectional view of FIG. 27(a) taken along line D-D, and FIG. 27(c) is an explanatory view showing another example.

FIG. 28 (a) is a plan view of a respiration sensor of Embodiment 17, FIG. 28(b) is a sectional view thereof and FIG. 28(c) is a bottom view thereof.

### DESCRIPTION OF SYMBOLS

1, 61, 81, 101, 111, 121, 161, 171, 201, 221, 241, 251, 261, 291: respiration sensor
3, 63, 71, 83, 91, 103, 113, 123, 163, 173, 219: sensor body
9, 87, 131, 215: connecting portion
13, 15, 127, 129, 179, 171, 207, 209, 225, 227: leg (side portion)
17, 191, 203, 223, 243, 253, 263, 281, 295: substrate (cover)
11, 67, 75, 85, 93, 115, 125, 165, 175: respiration detecting section
19, 69, 79, 97, 98, 117, 137, 139, 167, 169, 200, 217, 235, 247, 255, 279, 283, 293: piezoelectric element
25: piezoelectric body (PVDF film)
27, 29: electrode
68, 77, 78, 95, 96, 133, 135: inclining surface
65, 14: shield layer

### BEST MODE FOR CARRYING OUT THE INVENTION

A description will be given of the best mode for carrying out the present invention (embodiments).

### Embodiment 1

A respiration sensor of the present embodiment is to detect breathing of a patient and others.

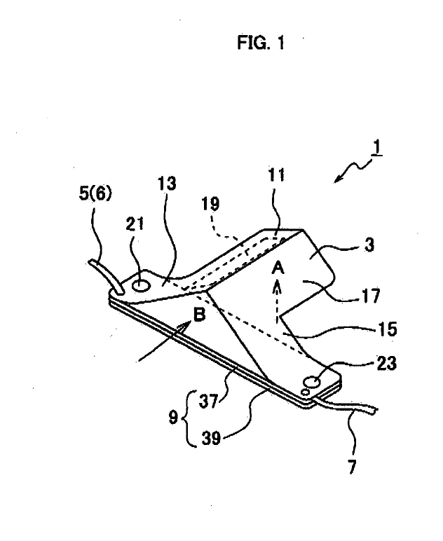

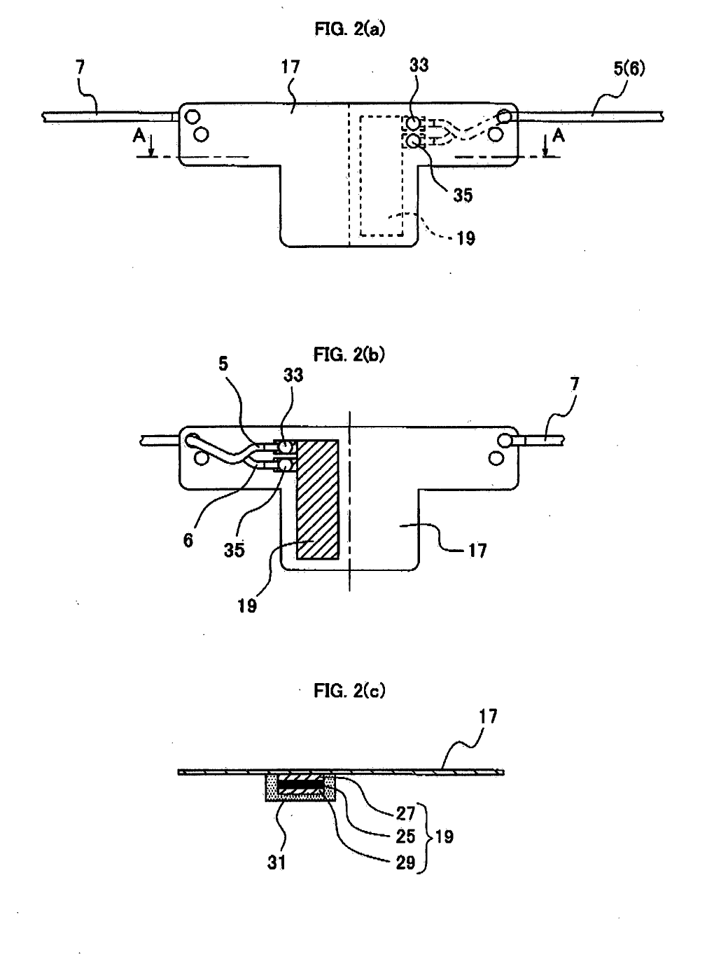

a) First, a description will be given of a constitution of the respiration sensor of the present embodiment by referring to FIG. 1, FIGS. 2 (a), (b) and (c).

As shown in FIG. 1, the respiration sensor 1 of the present embodiment mainly comprises a sensor body 3 which is formed approximately in a T shape, a pair of lead wires 5 and 6 extended from the left end of the sensor body 3 (being superimposed to give one line), a dummy wire 7 extended from the right end of the sensor body 3, and a connecting portion 9 for connecting the lateral lower ends of the sensor body 3.

The sensor body 3 is a film-like thin member which is folded chevronwise at the central part (line-symmetry axis of the sensor body 3; ridge line portion of the chevron) and which comprises a respiration detecting section 11 for detecting breath from a mouth and a nose and a pair of legs 13 and 15 protruded from both ends of the rear end side (at the lower left in the drawing) of the respiration detecting section 11. The sensor body 3 includes a film-like substrate (cover) 17 made with, for example, polyester approximately in a T shape and a rectangular piezoelectric element 19 stuck to the inside of one inclining surface of the chevron (at the lower side in the drawing; on the living body side).

Of these constitutions, the respiration detecting section 11 has a rectangular shape folded along the symmetrical axis at an angle of about 90° to 120° to project upward to form a chevron and arranged so as to oppose a breathing direction of a mouth (arrow A direction) for blocking the flow of breath from a mouth in contact therewith and also so as to be parallel with a breathing direction of a nose (arrow B direction).

Further, a pair of legs 13 and 15 are formed in a rectangular shape, the leading ends of which are fixed to both ends of the connecting portion 9 by crimping fittings 21 and 23.

As the sensor body 3 is developed and shown in FIGS. 2 (a), (b) and (c), the piezoelectric element 19 is provided with electrodes 27 and 29 on both vertical faces of a rectangular PVDP film 25, which is a piezoelectric body. These electrodes 27 and 29 are formed by subjecting a metal layer such as Ag, Pt, Au, Ni or the like to deposition, thick film printing or foil stamping. It is noted that the outside of the electrodes 27 and 29 is covered by a moisture-proof film 31.

Further, a pair of lead wires 5 and 6 are connected to the electrodes 27 and 29 respectively by crimping terminals 33 and 35. In other words, one lead wire 5 is connected by the crimping terminal 33 to the upper electrode 27 shown in FIG. 2(c), whereas the other lead wire 6 is connected by the crimping terminal 35 to the lower electrode 29 in FIG. 2 (c). It is noted that both electrodes 27 and 29 are arranged so as not to be in contact with each other.

Further, as shown in FIG. 1, the connecting portion 9 is a strip-shaped member, and includes a long supporting plate 37 made with an elastic material such as polyurethane and a double-sided tape (adhering portion) 39 adhered on a back side of the supporting plate 37.

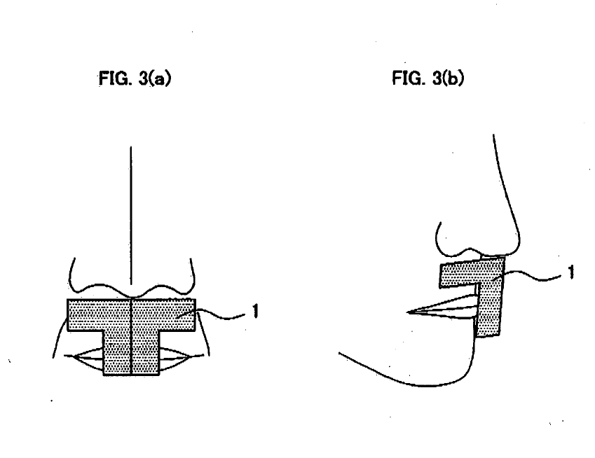

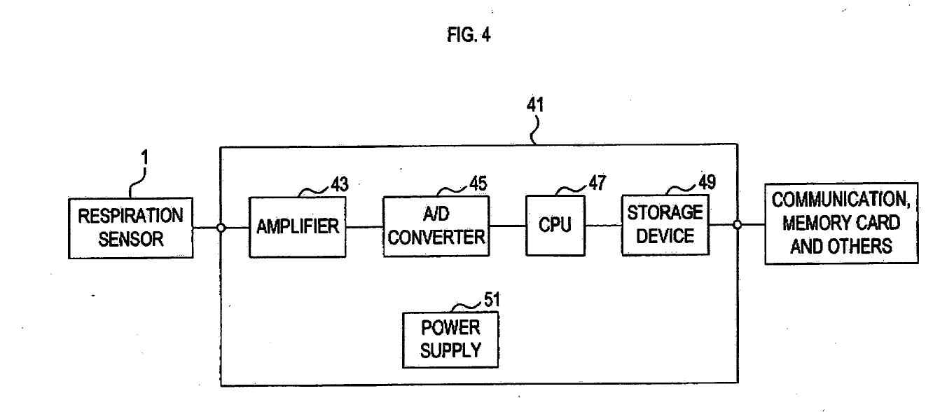

b) Next, a description will be given of a method for using the respiration sensor 1 by referring to FIGS. 3 (a) and (b) and FIG. 4.

As shown in FIGS. 3 (a) and (b), the respiration sensor 1 is stuck between a mouth and a nose. In other words, regarding the approximately T shaped respiration sensor 1, the double-sided tape 39 of the connecting portion 9 (corresponding to the horizontally extending portion of the T shape) is stuck between a mouth and a nose and arranged so that the respiration detecting section 11 (corresponding to the vertically extending portion of the T shape) is placed over a mouth.

Thereby, the piezoelectric element 19 of the respiration detecting section 11 is arranged apart from a mouth. Further, breath from a mouth comes into contact with the respiration detecting section 11 at an angle close to a right angle (a right angle on the symmetrical axis) and breath from a nose flows parallel therewith.

Then, when breathing is detected, lead wires 5 and 6 of the respiration sensor 1 are connected to a respiration state monitor 41 shown in FIG. 4.

The respiration state monitor 41 comprises a known amplifier 43, an A/D converter 45, a CPU 47, a storage device (backup RAM and others) 49, a power supply 51 and others. Memory cards and others are also attached to the respiration state monitor 41 in a removable manner. Further, the respiration state monitor 41 can send out measurement data and others through wire (or wireless) transmission.

c) Next, a description will be given of the effects of the present embodiment.

In the present embodiment, when breath from a mouth is brought into contact with the respiration detecting section 11 at a substantially perpendicular angle, the piezoelectric element 19 is deflected (distorted). Then, a voltage resulting from the distortion can be detected by the respiration state monitor 41, by which breath from a mouth can also be detected.

Further, since the PVDF of the piezoelectric element 19 shows a pyroelectric effect, it also responds to changes in temperature. Therefore, the PVDF can detect changes in temperature in association with breathing, thereby improving the accuracy on detection of breathing. In other words, although breath from a nose flows parallel with the piezoelectric element 19, the pyroelectric effect of the piezoelectric element 19 makes it possible to detect breath from a nose as well.

Still further, in the present embodiment, since the piezoelectric element 19 is arranged apart from the living body, there is provided an advantage that it is less likely to be influenced by the living body (other than breathing), thereby improving measurement accuracy.

It is noted that the respiration sensor 1 of the present embodiment is connected to the respiration state monitor of Embodiment 8, which will be described later, thereby making it possible to detect not only breathing but also snoring.

### Embodiment 2

Next, a description will be given of Embodiment 2. However, description of the contents similar to those of Embodiment 1 will not be repeated.

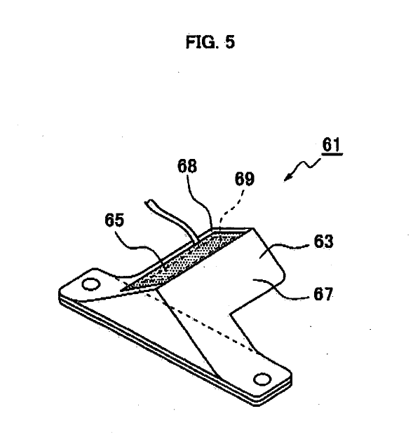

As shown n FIG. 5, a respiration sensor 61 of the present embodiment is provided with an electrically-conductive grounded shield layer 65 on a surface of the sensor body 63.

In other words, in the present embodiment, an piezoelectric element 69 is arranged inside one inclining surface 68 of a respiration detecting section 67 which has become a chevron shape, and a rectangular shield layer 65 is arranged on the outer face (upper face) of the inclining surface 68 so as to cover the entire surface of the piezoelectric element 69.

In the present embodiment, the shield layer 65 is provided, by which it is possible to prevent the respiration sensor 61 from electrostatic charge and to effect an electromagnetic wave shield. As a result, it is also possible to prevent a decrease in measurement accuracy due to noise.

### Embodiment 3

Next, a description will be given of Embodiment 3. However, description of the contents similar to those of Embodiment 1 will not be repeated.

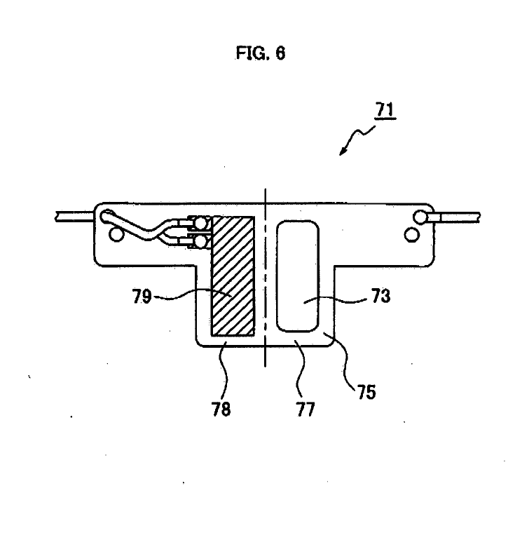

As shown in FIG. 6 (the sensor body is developed), a respiration sensor of the present embodiment is provided with an opening portion 73 on an inclining surface of a sensor body 71.

In other words, a rectangular piezoelectric element 79 is arranged on one inclining surface 77 of a respiration detecting section 75 which is to be a chevron shape, and a similar rectangular opening portion 73 is formed on the other inclining surface 78.

Thereby, breath from a mouth easily flows out via the opening portion 73 and breath from a mouth also easily comes into contact with the piezoelectric element 79 perpendicularly. There is provided an advantage that the measurement accuracy is improved.

### Embodiment 4

Next, a description will be given of Embodiment 4. However, description of the contents similar to those of Embodiment 1 will not be repeated.

As shown in FIG. 7, a respiration sensor 81 of the present embodiment is foldable at a central part.

In other words, a groove or the like at which the respiration sensor 81 is foldable inside is formed at a respiration detecting section 85 of a sensor body 83 and at a central part of a connecting portion 87 (a symmetrical axis of the respiration sensor 81 which is symmetrical). It is noted that the connecting portion 87 is folded so as to project inwardly.

Thereby, the respiration sensor 81 can be folded to make it compact at the time of shipment or packaging. As a result, there is provided an advantage that it is conveniently shipped or packed.

### Embodiment 5

Next, a description will be given of Embodiment 5. However, description of the contents similar to those of Embodiment 1 will not be repeated.

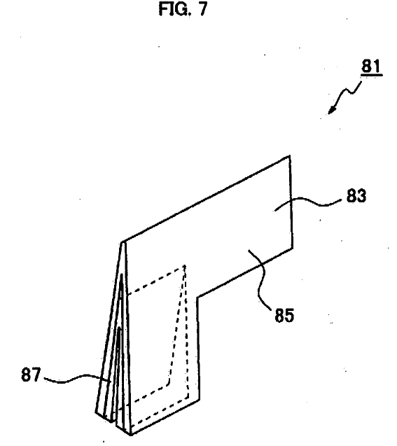

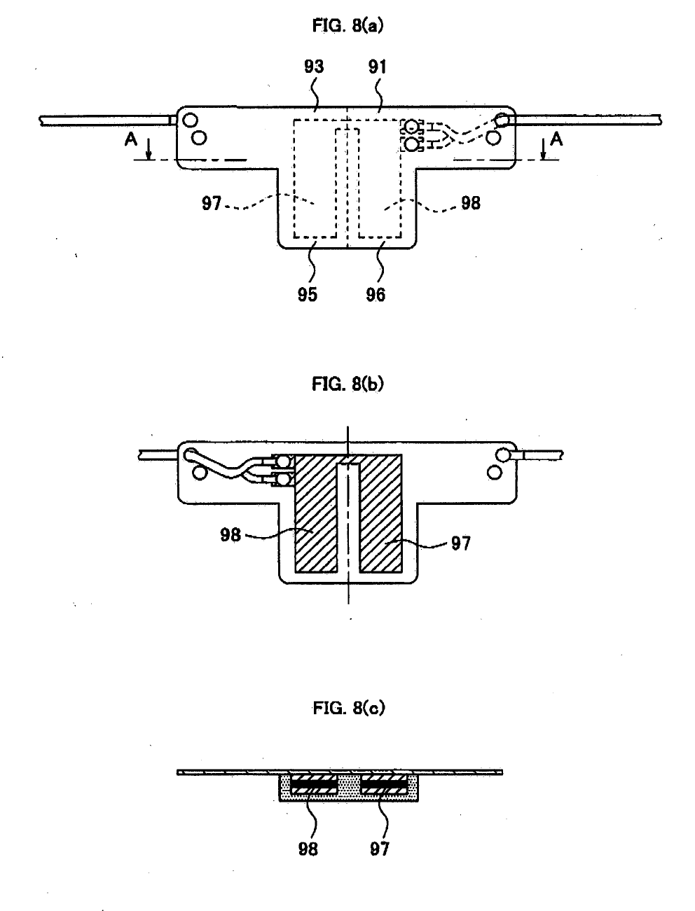

As a sensor body is developed and shown in FIGS. 8 (a), (b) and (c), in a respiration sensor of the present embodiment, piezoelectric elements 97 and 98 are respectively arranged at the left and right inclining surfaces 95 and 96 of a respiration detecting section 93, which are to be a chevron shape on a sensor body 91.

Thereby, there is provided an advantage that breathing can be detected more effectively to increase measurement accuracy.

### Embodiment 6

Next, a description will be given of Embodiment 6. However, description of the contents similar to those of Embodiment 1 will not be repeated.

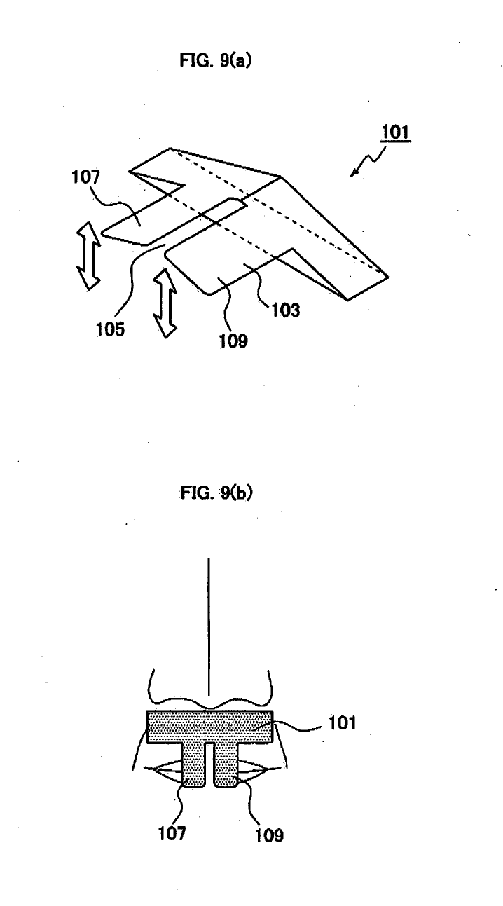

As shown in FIG. 9 (a), in a respiration sensor 101 of the present embodiment, a slit 105 is provided at the central part of the sensor body 103 to form two tabs 107 and 109.

In other words, piezoelectric elements (not shown) are arranged respectively at the two tabs 107 and 109 extending to a leading end side (at the lower left in the drawing) of the sensor body 103, by which each of the tabs 107 and 109 can be deflected easily in a vertical direction.

Thereby, in the present embodiment, as shown in FIG. 9 (b), where the tabs 107 and 109 are arranged over a mouth, they can be greatly deflected corresponding to breath from a mouth. Thus, there is provided an advantage that the measurement accuracy is improved.

### Embodiment 7

Next, a description will be given of Embodiment 7. However, description of the contents similar to those of Embodiment 1 will not be repeated.

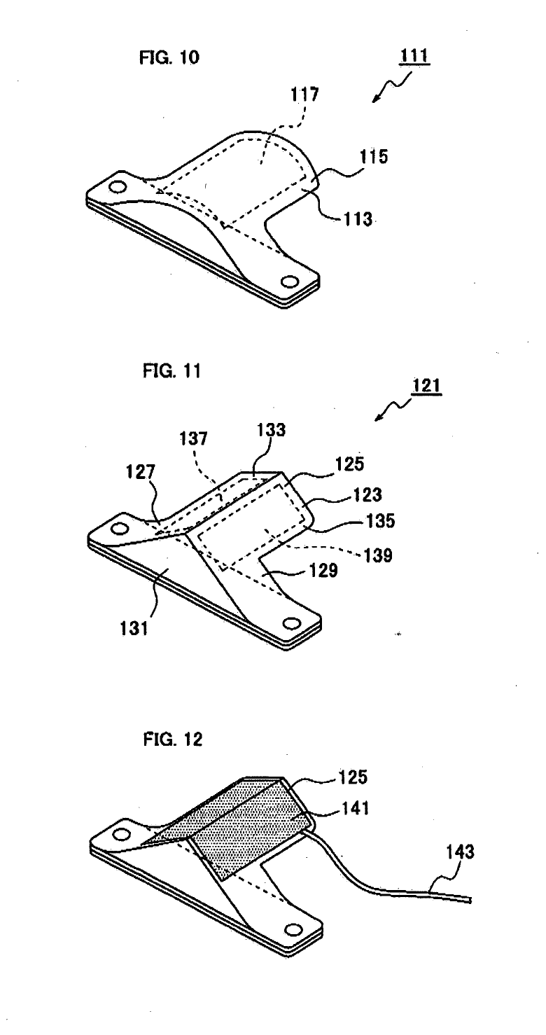

As shown in FIG. 10, in a respiration sensor 111 of the present embodiment, a sensor body 113 is curved in an R shape, and a piezoelectric element 117 similarly curved in an R shape is arranged at a central part of a respiration detecting section 115.

The present embodiment also offers the effects similar to those of the previously described Embodiment 1. Further, since the respiration detecting section 115 is formed in an R shape, there is provided an advantage that it can be easily deflected to improve measurement accuracy.

### Embodiment 8

Next, a description will be given of Embodiment 8. However, description of the contents similar to those of Embodiment 1 will not be repeated.

The present embodiment is a respiration sensor capable of detecting not only normal breathing but also snoring.

As shown in FIG. 11, a respiration sensor 121 of the present embodiment is provided with a sensor body 123 bent chevronwise and a connecting portion 131 for connecting both legs 127 and 129 extending from a respiration detecting section 125 of the sensor body 123.

A first piezoelectric element 137 for detecting breathing is arranged inside one inclining surface 133 of the respiration detecting section 125, whereas a second piezoelectric element 139 for detecting snoring is arranged inside the other inclining surface 135.

Further, as shown in FIG. 12, an electrically-conductive shield layer (for example, aluminum foil) 141 is formed outside the respiration detecting section 125 so as to cover both piezoelectric elements 137 and 139. It is noted that the shield layer 141 is grounded by a lead wire 143.

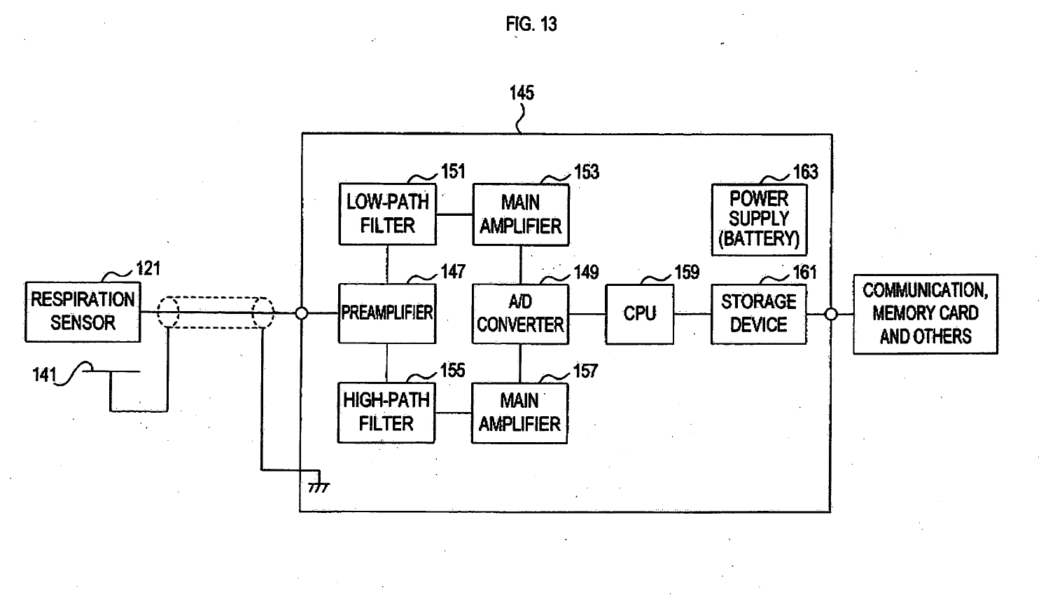

Then, as shown in FIG. 13, the above-described respiration sensor 121 is connected to a respiration state monitor 145. The respiration state monitor 145 is provided with a low-pass filter 151 and a main amplifier 153 between a preamplifier 147 and an A/D converter 149. It is also provided with a high-pass filter 155 and a main amplifier 157. It is additionally provided with a CPU 159, a storage device 161, a power supply 163 and others. Further, a memory card and the like can be attached to the respiration state monitor 145 in a removable manner. It is noted that the respiration state monitor 145 can send out measurement data and the like by wire (or wireless) transmission.

Of these components, the low-pass filter 151 and the high-pass filter 155 are used to detect snoring.

In other words, in the case of normal breathing motions (without snoring), the respiration sensor 121 outputs signals at a low frequency, whereas in the case of snoring, the respiration sensor 121 outputs signals at a high frequency. Thus, the low-pass filter 151 and the high-pass filter 155 are used to identify the magnitude of frequency of a signal output from the respiration sensor 121, thereby making it possible to detect snoring.

Therefore, the present embodiment offers the effects similar to those of the previously described Embodiment 1. There is provided a remarkable effect that the present embodiment can detect not only normal breathing but also snoring.

Since the present embodiment is provided with a grounded shield layer 141, it is possible to prevent electrostatic charge of the respiration sensor 121. Further, an electromagnetic wave shield is effected on the shield layer 141, thereby making it possible to prevent a decrease in measurement accuracy due to noise.

### Embodiment 9

Next, a description will be given of Embodiment 9. However, description of the contents similar to those of Embodiment 8 will not be repeated.

The present embodiment is a respiration sensor capable of detecting not only normal breathing but also snoring.

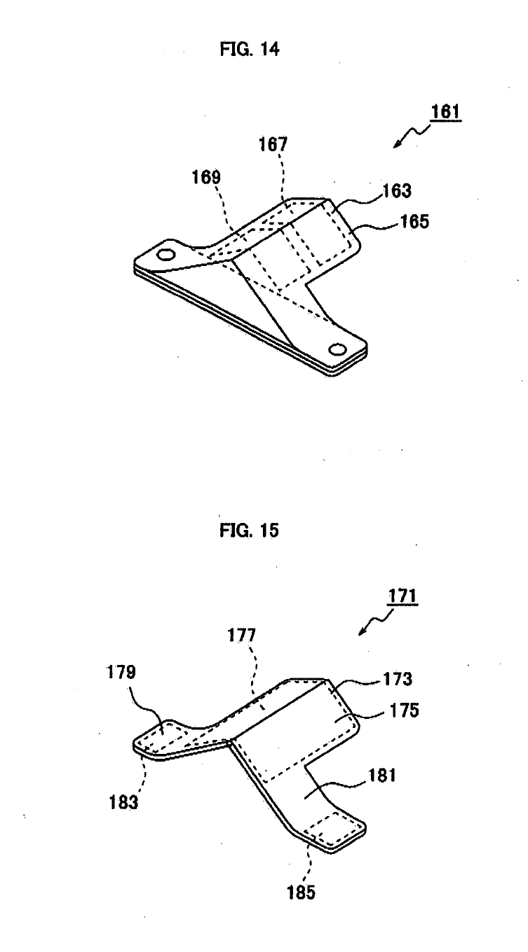

As shown in FIG. 14, as for a respiration sensor 161 of the present embodiment, a first piezoelectric element 167 for detecting breathing and a second piezoelectric element 169 for detecting snoring are provided at a respiration detecting section 165 of the sensor body 163, which is bent chevronwise sequentially from a leading end side thereof (the upper right side in the drawing) along a symmetrical axis which is the a of a chevron. It is noted that both piezoelectric elements 167 and 169 are bent chevronwise.

Therefore, the present embodiment offers the effects similar to those of the above-described Embodiment 8.

### Embodiment 10

Next, a description will be given of Embodiment 10. However, description of the contents similar to those of Embodiment 1 will not be repeated.

The respiration sensor of the present embodiment is not provided with a connecting portion and simple in constitution.

As shown in FIG. 15, a respiration sensor 171 of the present embodiment is such that a sensor body 173 is folded chevronwise. One unit of a piezoelectric element 177 (which is folded chevronwise) is arranged on the substantially entire surface at a respiration detecting section 175, and legs 179 and 181 are extended from a respiration detecting section 175 in a lateral direction.

Legs 179 and 181 are slightly folded outside at the leading end, and double-sided tapes 183 and 185 are stuck on the inner faces thereof.

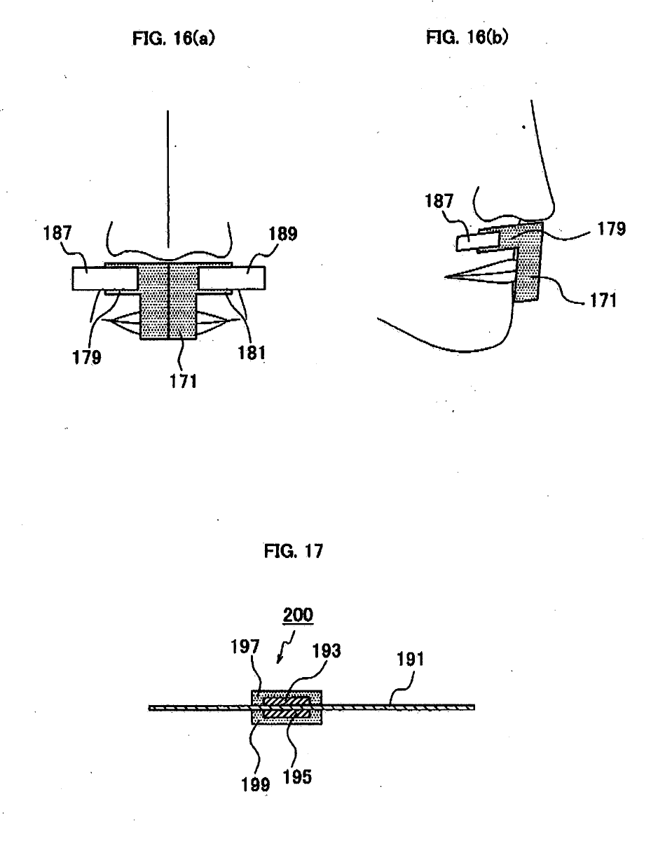

As shown in FIGS. 16(a) and (b) in the present embodiment, the leading ends of the legs 179 and 181 are pressed between a mouth and a nose and adhered by using double-sided tapes 183 and 185. Further, as shown in the drawings, it is also acceptable to stick adhesive tapes 187 and 189 from outside the legs 179 and 181 and fix the respiration sensor 171. In this instance, the double-sided tapes 183 and 185 can be saved.

In the present embodiment, no connecting portion is provided to simplify the constitution and reduce costs. Further, the absence of the connecting portion will not interfere with a moustache when the respiration sensor is stuck thereon. This is advantageous to those having a moustache.

### Embodiment 11

Next, a description will be given of Embodiment 11. However, description of the contents similar to those of Embodiment 1 will not be repeated.

Unlike Embodiment 1 in which a piezoelectric element is stuck on the surface of a film-like substrate, in the respiration sensor of the present embodiment, as the cross section thereof is developed and shown in FIG. 17, a PVDF film (piezoelectric body), which is an insulator, is used to form electrodes 193 and 195 similar in shape to the electrode of Embodiment 1 on both faces of the film, and the electrodes are covered with moisture-proof films 197 and 199.

In this instance, portions of the piezoelectric body, which form the electrodes 193 and 195 (first portions), act as a piezoelectric element 200, and the other portions (second portions) act as a substrate 191 for retaining a piezoelectric element 200 at a predetermined detection position.

### Embodiment 12

Next, a description will be given of Embodiment 12. However, description of the contents similar to those of Embodiment 1 will not be repeated.

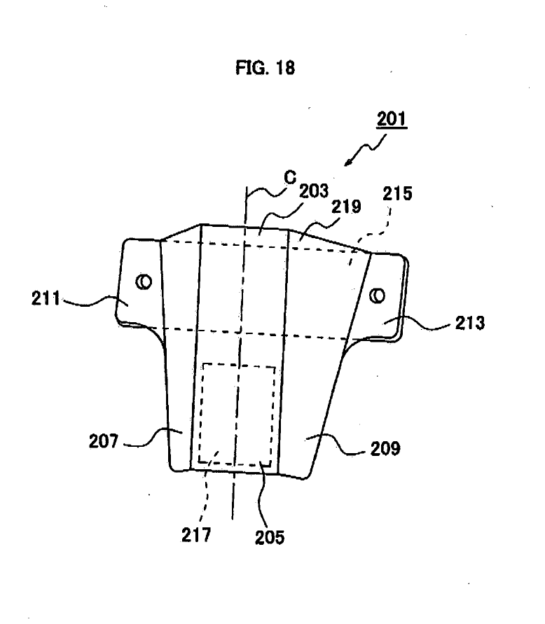

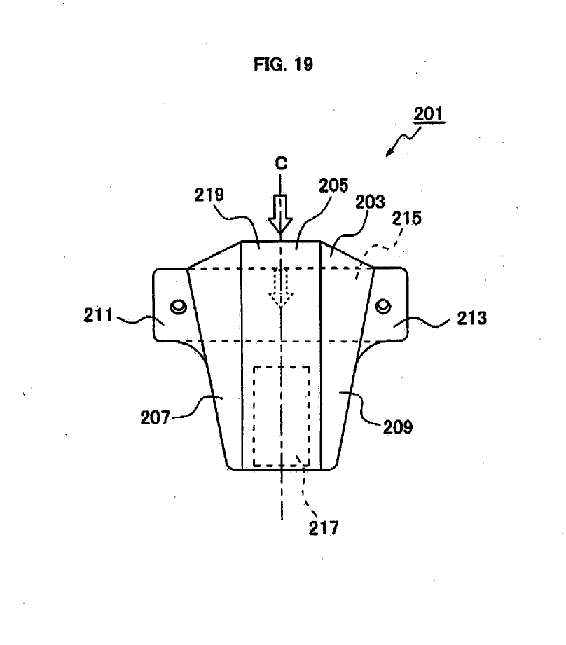

As shown in FIG. 18 and FIG. 19, a respiration sensor 201 of the present embodiment is such that a substrate 203, which is a film-like cover, is folded so as to protrude outward at a central part thereof along one axis (one axis along the breathing direction of a nose; C axis).

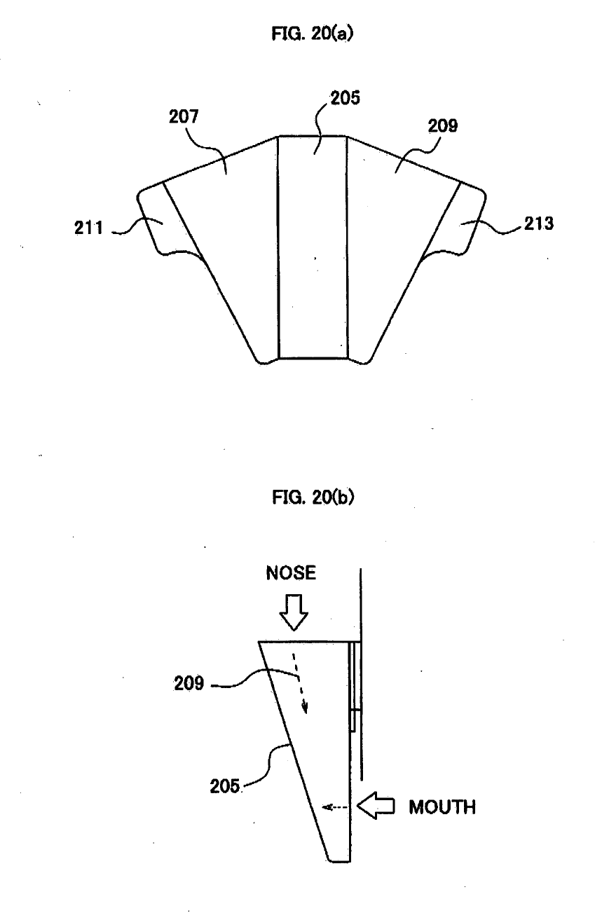

Specifically, the substrate 203 comprises a strip-shaped central part 205 extending along the C axis at the center, side portions (legs) 207 and 209 which are approximately triangular in shape and folded inward (to the living body side) on both sides of the central part 205, with the leading end made narrower in width, and feet 211 and 213 which are folded outward on both sides of the rear end side (the nose side) at each of the side portions 207 and 209 (refer to the developed view of FIG. 20 (a)).

A concave portion, which is inside the thus folded substrate 203, is formed so that the perpendicular cross section with respect to the C axis is trapezoidal and the leading end side is made smaller in the cross section area than the rear end side. In other words, the concave groove inside the substrate 203 is constituted so that a flow path of breath from a nose is converged to the leading end side and breath from a mouth is collected at the central part 205 side (refer to the side view of FIG. 20 (b)).

Further, a film-like piezoelectric element 217 is stuck on the inside of the leading end side the central part 205. In this instance, a sensor body 219 is constituted from the piezoelectric element 217 and the substrate 203.

It is noted that a connecting portion 215 is formed so as to connect left and right feet 211 and 213. The bottom face side of the connecting portion 215 is stuck between a mouth and a nose.

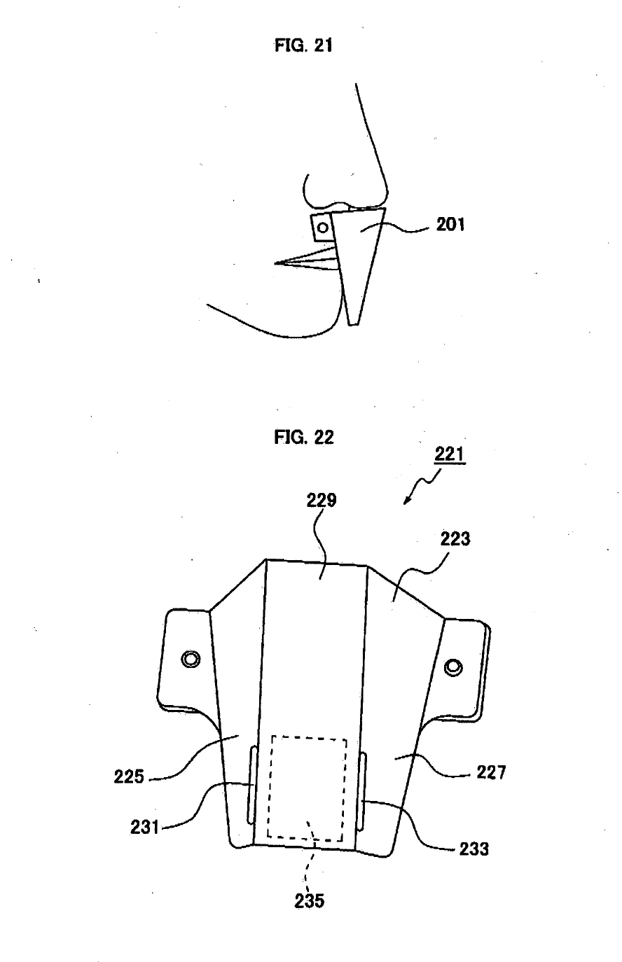

In the present embodiment, the substrate 203, which is a film-like cover folded so as to make the living body side concave, is used. Therefore, as shown in FIG. 21, where the respiration sensor 201 is stuck between a nose and a mouth, it is possible to guide breath from a nose and breath from a mouth effectively to the piezoelectric element 217 side.

In particular, a groove inside the substrate 203 is trapezoidal in the cross section, and an area of the cross section is set to be smaller as it is closer to the leading end side. As a result, it is possible to guide effectively breath from a nose and breath from a mouth to the piezoelectric element 217 side.

Thereby, there is provided a remarkable advantage that measurement accuracy is very high.

### Embodiment 13

Next, a description will be given of Embodiment 13. However, description of the contents similar to those of Embodiment 12 will not be repeated.

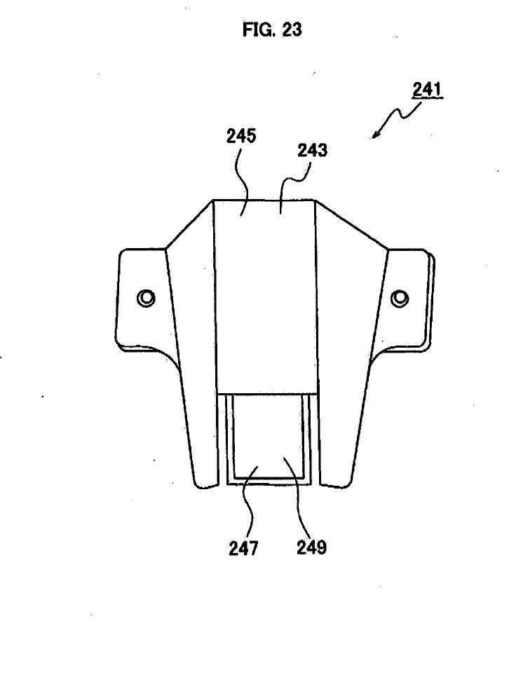

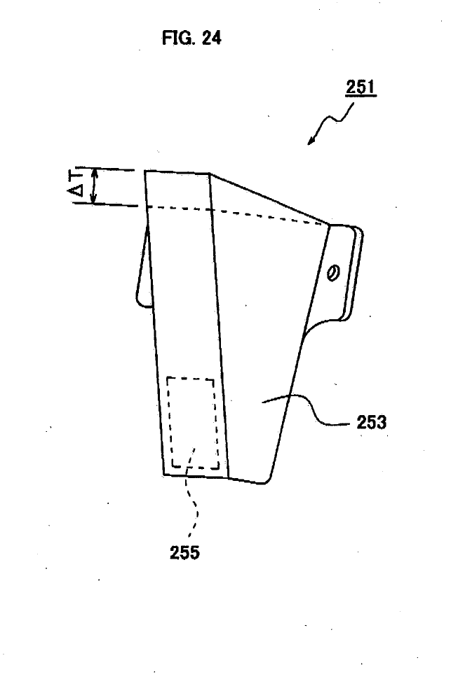

As shown in FIG. 22, a substrate 223, which is a cover of a respiration sensor 221 in the present embodiment, is fundamentally similar in shape to that of the above described Embodiment 12.

In the present embodiment, slit-like ventilation holes 231 and 233 are formed along a central part 229 side at the leading end side of side portions 225 and 227, that is, at a portion where a central part 229 and the side portions 225 and 227 are folded. More specifically, a pair of left and right ventilation holes 231 and 233 are formed in a lateral direction of a piezoelectric element 235.

Thereby, after breath from a nose or a mouth is guided to the piezoelectric element 235 side, it is easily discharged outside via these ventilation holes 231 and 233, thereby eliminating an excessive retention of breath in the vicinity of the piezoelectric element 235. As a result, a temperature of the piezoelectric element 235 is inhibited from being excessively increased due to breath, thus making it possible to accurately detect the presence or absence of breathing and snoring.

### Embodiment 14

Next, a description will be given of Embodiment 14. However, description of the contents similar to those of Embodiment 12 will not be repeated.

As shown in FIG. 23, a substrate 243, which is a cover of a respiration sensor 241 in the present embodiment, is fundamentally similar in shape to that of the above described Embodiment 12.

In the present embodiment, a leading end of a central part 245 is cut, by which a piezoelectric element 247 is exposed outside.

For this reason, after breath from a nose or a mouth is guided to the piezoelectric element 247 side, it is easily discharged outside via the thus cut portion 249, thereby eliminating an excessive retention of breath in the vicinity of the piezoelectric element 247. As a result, a temperature of the piezoelectric element 247 is inhibited from being excessively increased due to breath, thus making it possible to accurately detect the presence or absence of breathing and snoring.

Further, since the piezoelectric element 247 is formed in a cantilever shape, there is provided an advantage that it can be easily deflected when breath is brought into contact therewith to provide a large sensor output.

### Embodiment 15

Next, a description will be given of Embodiment 15. However, description of the contents similar to those of Embodiment 12 will not be repeated.

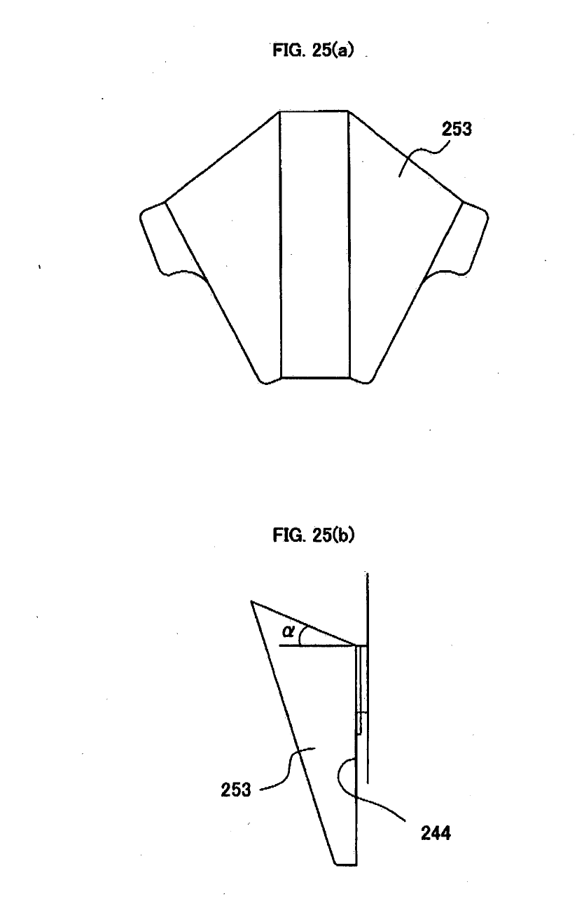

As shown in FIGS. 24 and FIG. 25 (a), a substrate 243, which is a cover of a respiration sensor 251 in the present embodiment, has a shape protruding outward, that is, fundamentally similar in shape to that of the above described Embodiment 12.

In particular, in the present embodiment, a rear end side of the central part 245 is protruded largely (for example, ΔT) to a nose side. In other words, as shown in FIG. 25 (b), the nose side of a substrate 243 is not perpendicular to a bottom face 244 of the substrate 243 but protruded, with an inclination to the nose side only by a predetermined angle of α.

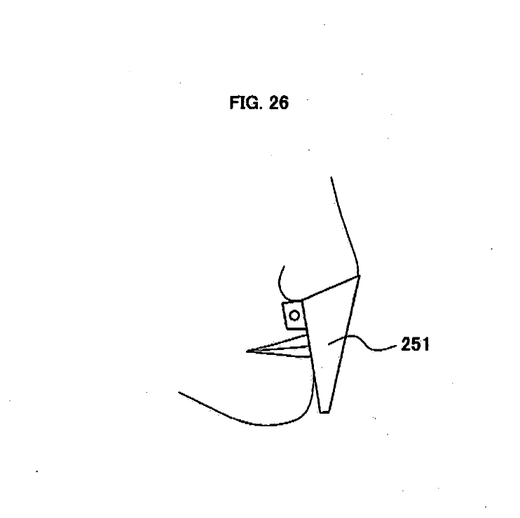

Therefore, as shown in FIG. 26, when the respiration sensor 251 is stuck between a nose and a mouth, the nostrils can be covered without any space by the substrate 243 greatly overhanging to the nose side.

For this reason, since breath from a nose can be guided in particular effectively to the piezoelectric element, there is provided an effect that the measurement accuracy is improved.

### Embodiment 16

Next, a description will be given of Embodiment 16. However, description of the contents similar to those of Embodiment 13 will not be repeated.

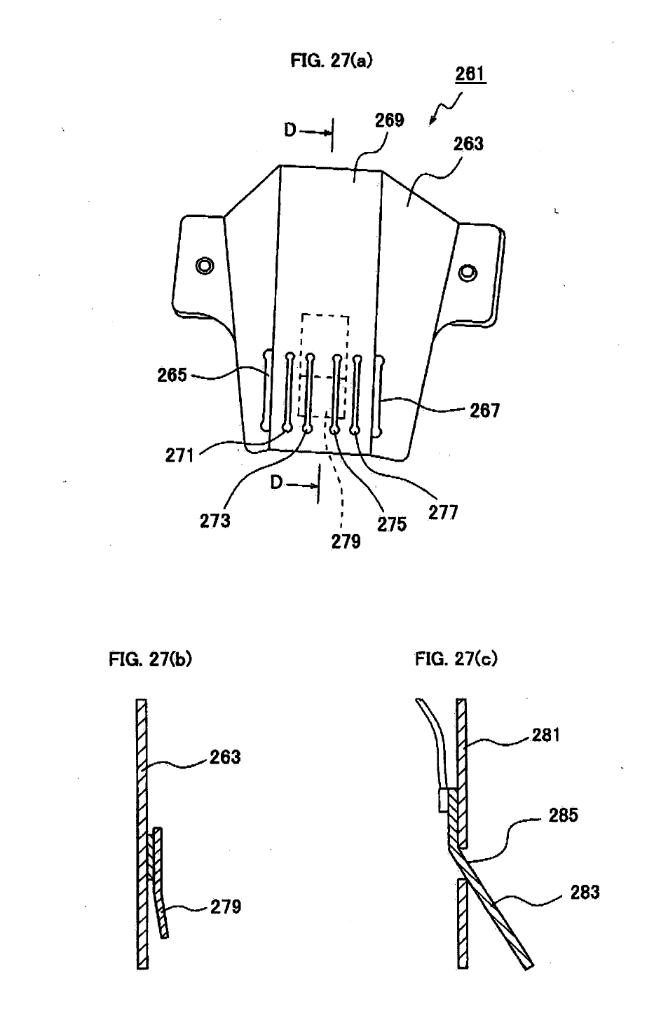

As shown in FIG. 27(a), a substrate 263, which is a cover of a respiration sensor 261 in the present embodiment, is fundamentally similar in shape to that of the above described Embodiment 12.

The present embodiment is provided at a central part 269 with slit-like ventilation holes 271 to 277 in four arrays along a longitudinal direction, in addition to ventilation holes 265 and 267 similar to those of Embodiment 12.

In other words, the ventilation holes 271 to 277 are formed outside of a piezoelectric element 279 at a leading end side of a central part 269 (a projection portion with respect to the central part 269).

Further, as shown in FIG. 27(b), the piezoelectric element 279 is folded inward (to the living body side) so that the leading end side is raised.

Thereby, after breath from a nose or a mouth is guided to the piezoelectric element 235 side, it is easily discharged outside via these ventilation holes 265 to 277, thereby eliminating an excessive retention of breath in the vicinity of the piezoelectric element 279. As a result, a temperature of the piezoelectric element 279 is inhibited from being excessively increased due to breath, thus making it possible to accurately detect the presence or absence of breathing and snoring.

Further, in the present embodiment, a leading end of the piezoelectric element 279 is bent inward, by which an inner surface on the leading end side of the piezoelectric element 279 is opposed to a breathing direction of a mouth and also opposed to a breathing direction of a nose. In other words, the inner surface at the leading end side of the piezoelectric element 279, a breathing direction of a mouth and a breathing direction of a nose are not parallel with the piezoelectric element 279 but inclined respectively at an angle greater than a predetermined angle (an angle exceeding 0°, for example, 10° or greater). Therefore, there is provided an advantage that measurement accuracy is made higher.

It is noted that, as another example, as shown in FIG. 27 (c), a leading end of a piezoelectric element 283 is not bent inside a substrate 281, but the piezoelectric element 283 may be attached by inserting it at a certain angle via an opening portion 285 outside the substrate 281.

### Embodiment 17

Next, a description will be given of Embodiment 17. However, description of the contents similar to those of Embodiment 1 will not be repeated.

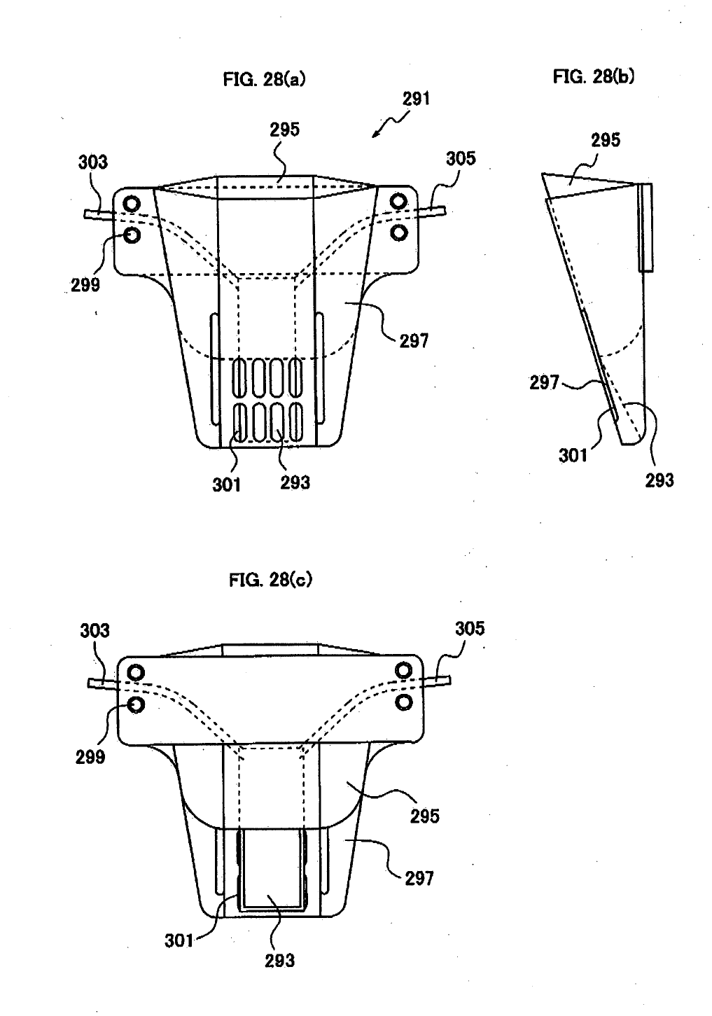

As shown in FIGS. 28 (a), (b) and (c), a respiration sensor 291 of the present embodiment is double-structured by a substrate 295 at which a piezoelectric element 293 is arranged and an outer cover 297 which covers the outside thereof.

In other words, the piezoelectric element 293 is attached to a leading end of the substrate 295 (which is approximately in a T shape on development), and the outer cover 297 (which is approximately in a T shape on development) is arranged so as to cover an outside of the piezoelectric element 293 and the substrate 295 at a predetermined clearance.

Then, both left and right sides of the substrate 295 (refer to FIG. 28 (a)) and both left and right sides of the outer cover 297 are fixed by an eyelet 299 and made into an integral form.

Further, a plurality of slit-like ventilation holes 301 are formed on the outer cover 297 corresponding to the outside of the piezoelectric element 293.

Further, lead wires 303 and 305 extending from an upper end side of the piezoelectric element 293 given in the above drawing are pulled around into a clearance between the substrate 295 and the outer cover 297 therefor, and pulled out from a space between a pair of eyelets 299 arranged on both left and right sides of the substrate 295 and the outer cover 297.

It is noted that the leading end side of the piezoelectric element 293 is folded inward from the upper face of the substrate 293, for example, at about 15°.

Therefore, in the present embodiment, since the piezoelectric element 293 is covered by the outer cover 297, there is provided an effect that it is not touched inadvertently from outside by fingers, etc. The lead wires 303 and 305 extending from the piezoelectric element 293 are arranged between the substrate 295 and the outer cover 297, thereby there is provided an effect that the lead wires 303 and 305 are not obstructive.

It is noted that the present invention is not in any way restricted to the embodiments so far described, and, as a matter of course, the present invention may be executed in various modes as long as it is not deviated from the technical scope of the present invention.

(1) For example, in place of using an integrally-fabricated piezoelectric element, separate piezoelectric elements may be used and connected electrically.

(2) Further, a shield layer may be used to cover only the upper face of the respiration sensor, only the lower face thereof or both upper and lower faces.

(3) Still further, for example, where piezoelectric elements are respectively arranged on the left and right inclining surfaces which have become a chevron shape, one piezoelectric element may be different in thickness from the other piezoelectric element. Thereby, degree of deflection or responsiveness can be changed to provide an advantage that an optimal state is realized in detecting breathing and snoring.

1. A respiration sensor to be attached on a surface of a living body for detecting a respiration state of the living body, comprising:
a detecting element for outputting a signal corresponding to at least one of the respiration states of breathing and snoring of the living body; and
a substrate for retaining the detecting element at a predetermined detection position apart from the surface of the living body,
the detecting element being arranged so that breath from both a mouth and a nose is brought into contact therewith.

2. The respiration sensor as set forth in claim 1, wherein the detecting element is a piezoelectric element or a temperature sensing element.

3. The respiration sensor as set forth in claim 1, wherein the detecting element is arranged so as to oppose a breathing direction of a mouth and also extend along a breathing direction of a nose.

4. The respiration sensor as set forth in claim 1, wherein the detecting element is arranged so that breath at least either from a mouth or a nose is brought into contact therewith at an angle greater than a predetermined angle.

5. The respiration sensor as set forth in claim 1, wherein the substrate is configured so that breath at least either from a mouth or a nose is guided to a detecting element side.

6. The respiration sensor as set forth in claim 1, wherein an inside of the substrate, which is the living body side, is shaped in a concave form.

7. The respiration sensor as set forth in claim 6, wherein the detecting element is arranged on a bottom surface of a concave groove of the substrate.

8. The respiration sensor as set forth in claim 6, wherein the concave groove is formed so that a cross section perpendicular to a flow path of breath from a nose gives a trapezoidal shape.

9. The respiration sensor as set forth in claim 6, wherein the concave groove is formed so that the farther an area of the cross section perpendicular to the flow path of breath from a nose is from a nose side, the smaller it is.

10. The respiration sensor as set forth in claim 6, wherein the nose side of the substrate protrudes further to the nose side than a position at which the substrate is attached to the living body along the flow path of breath from a nose on the concave groove.

11. The respiration sensor as set forth in claim 1, wherein the substrate is a plate-like member.

12. The respiration sensor as set forth in claim 11, wherein the substrate is provided with a surface which is opposed to a breathing direction of a mouth and also extends along a breathing direction of a nose.

13. The respiration sensor as set forth in claim 1, wherein the detecting element is formed in a plate shape and includes a surface opposing a breathing direction of a mouth and also extending along a breathing direction of a nose.

14. The respiration sensor as set forth in claim 1, wherein the detecting element is arranged on a surface of the substrate.

15. The respiration sensor as set forth in claim 14, wherein the detecting element is arranged on the living body side of the substrate.

16. The respiration sensor as set forth in claim 1, wherein where the detecting element is a piezoelectric element, a ventilation hole is formed in the substrate in accordance with a position at which the piezoelectric element is arranged.

17. The respiration sensor as set forth in claim 16, wherein the ventilation hold is formed at a projection area of the detecting element to the substrate.

18. The respiration sensor as set forth in claim 16, wherein the ventilation hole is formed beside the detecting element.

19. The respiration sensor as set forth in claim 1, wherein where the detecting element is a piezoelectric element, the substrate is not provided outside the piezoelectric element but the piezoelectric element is exposed outside.

20. The respiration sensor as set forth in claim 1, comprising:
a plate-like piezoelectric body having a first portion and a second portion, wherein
the detecting element is constituted with the first portion of the piezoelectric body and electrodes installed on both sides of the first portion in a thickness direction, end
the substrate is constituted with the second portion of the piezoelectric body.

21. The respiration sensor as set forth in claim 1, wherein an opening portion penetrating through the substrate is formed in a face of the substrate opposing a breathing direction of a mouth,

22. The respiration sensor as set forth in claim 1, wherein a slit is provided along an exhaling direction from a leading end thereof at a portion of the substrate where the detecting element is arranged and which is positioned at the leading end side in the exhaling direction of a nose.

23. The respiration sensor as set forth in claim 1, wherein the substrate is folded or curved to keep a distance between the living body and the detecting element.

24. The respiration sensor as set forth in claim 1, wherein the substrate includes a leg for separating the detecting element from the surface of the living body.

25. The respiration sensor as set forth in claim 24, wherein an adhering portion for adhering the respiration sensor to the living body is provided at a leading end side of the leg on the living body side.

26. The respiration sensor as set forth in claim 25, comprising: a pair of left and right legs, and a connecting portion for connecting these legs and further comprising an adhering portion for adhering the respiration sensor to the living body, the adhering portion being provided on a living body side of the connecting portion,

27. The respiration sensor as set forth in claim 1, wherein a piezoelectric element for detecting breathing and a piezoelectric element for detecting snoring are arranged as the detecting element.

28. The respiration sensor as set forth in claim 27, wherein the piezoelectric element for detecting breathing is different in thickness from the piezoelectric element for detecting snoring.

29. The respiration sensor as set forth in claim 1, wherein where the detecting element is a piezoelectric element, an electrically-conductive shield layer is arranged so as to cover a surface of the piezoelectric element.

30. The respiration sensor as set forth in claim 1, wherein where the detecting element is a piezoelectric element, a leading end side of the piezoelectric element is bent toward the living body or the piezoelectric element is attached to the substrate at a certain angle.

31. The respiration sensor as set forth in claim 1, wherein the respiration sensor is foldable and developable.

32. The respiration sensor as set forth in claim 1, which is provided with a substrate for retaining the detecting element and an outer cover for covering the detecting element outside the substrate.

33. The respiration sensor set forth in claim 32, wherein a ventilation hole is formed on the outer cover.

34. A method for using the respiration sensor as set forth in claim 31, wherein the respiration sensor is folded at the time of shipment or packaging.

35. A method for using the respiration sensor as set forth in claim 1, wherein the respiration sensor is stuck between a mouth and a nose in using the respiration sensor.

36. A respiration state monitor for monitoring a respiration state of the living body by using a signal from the respiration sensor as set forth in claim 1, comprising:
an amplifier section for amplifying signals from the respiration sensor I and a storage device for storing the signals.

37. The respiration state monitor as set forth in claim 36 wherein a signal from the respiration sensor is identified by a frequency band to detect a breathing signal indicating breathing and a snoring signal indicating snoring.

38. The respiration state monitor as set forth in claim 36, wherein a portable storage device is attached in a removable manner.

39. The respiration state monitor as set forth in claim 36, which is provided with communication functions for transmitting data to an outside.
